(19) United States
(12) Patent Application Publication
(10) Pub. No.: US 2006/0079922 A1
(43) Pub. Date: Apr. 13, 2006

(54) Balloon anchored surgical apparatus, its use and manufacture

(76) Inventor: Brian Creston, Milford, CT (US)

Correspondence Address: Kimberly V. Perry, Patent & Trademark Counsel;U.S. Surgical, a division of Tyco Healthcare Group LP, 150 Glover Avenue, Norwalk, CT, 06856, US

(21) Appl. No.: 10/961,905

(22) Filed: Oct. 12, 2004

(51) Int. Cl. A61M 29/00

(52) U.S. Cl. 606/192; 606/167

(57) ABSTRACT

A surgical instrument is provided including a housing having an orifice; a cannula defining a lumen which is in communication with the orifice; an obturator receivable through the orifice and the lumen; and a balloon having a distal side, a proximal side and an aperture extending through the distal side and the proximal side. The surgical instrument further includes at least one attachment member to facilitate welding of the cannula and a to the balloon.

### BACKGROUND

The present disclosure relates generally to surgical instruments and, more particularly, to surgical instruments including access devices for maintaining an operating space within a cavity in a patient's body.

During laparoscopic procedures, cannulas are utilized to provide an access port for surgical instruments and a conduit for introducing insufflation gases into the body cavity. Typically, a trocar is positioned within the cannula and utilized to guide or advance the cannula into the tissue or abdominal wall. Thereafter, the trocar is removed leaving the cannula in place at which time insufflation gas may be forced into the body cavity to form an anatomical operating space. In certain instances, a dissection instrument having a dissection balloon operatively connected to a distal end thereof is inserted into the body cavity. The dissection balloon is inflated to separate the tissue. It is important that a fluid seal is maintained between the dissection balloon and the exterior of the body.

One known balloon dissector has an access cannula with a threaded stabilization device. The threaded stabilization device prevents the cannula from migrating further into or out through the incision. Additionally, the stabilization device also operates as a skin seal, to prevent leakage of insufflation gases.

Balloon anchors on access cannulas are generally known, such balloon anchors are disposed inside the body and inflated. A foam collar is utilized on the exterior of the access cannula to hold the cannula in place, in cooperation with the balloon anchor. The balloon also prevents leakage of insufflation.

Another prior art device, known as a structural balloon trocar (“SBT”), is used to maintain an operating space within a cavity of a the body. Such SBT may be used in hernia repair operations, to maintain the operating space and access a hernia. Like the Balloon anchored access cannulas, the SBT includes an insufflation port, for introducing insufflation gases to aid in maintaining the operating space. The SBT also has a foam collar for securing the device and sealing around the incision.

In each of the devices above, the balloons, which comprise either a polymeric or elastomeric material, are attached to a member, which is also typically polymeric. For example, in the SBT, the balloon is heat welded to a collar and the collar is attached to a tubular member utilizing an adhesive. The process requires multiple steps and separate equipment for attaching the balloon to the collar and then attaching the collar to the tubular member. Improved methods of manufacturing balloon devices are desired.

### SUMMARY

According to one aspect of the present disclosure, there is provided a surgical instrument including a housing having an orifice; a cannula having a proximal end connected to the housing and a distal end, the cannula having a lumen which is in communication with the orifice; an obturator receivable through the orifice and the lumen; and a balloon. The surgical instrument further includes one or more attachment members for securing the balloon to the cannula. The one or more attachment members are welded to the cannula, and the balloon is welded to the one or more attachment members. In one embodiment employing a single attachment member, the attachment member is a sleeve disposed on the outer surface of the cannula. In another embodiment employing two attachment members, the attachment members are a first collar and a second collar. The first and second collars each have a tube portion welded to the cannula and a flange welded to the balloon.

The one or more attachment members are fabricated from a material that is compatible with both the material of construction of the cannula and the material of construction of the balloon. In this manner the one or more attachment members permit the welding of otherwise incompatible materials. In a particularly useful embodiment, the cannula is made from a polycarbonate material and the surface of the balloon that is secured to the cannula is made from a polyurethane. In this embodiment, the one or more attachment materials are advantageously made from an aliphatic polycarbonate-based thermoplastic polyurethane.

The balloon may include a multilayer material having a first layer of a first polymeric material, a second layer of a second polymeric material and a third layer of a third polymeric material, the second layer being interposed between the first layer and the third layer. Desirably, the first and third polymeric materials comprise polyurethane and the second polymeric material comprises polyester. It is envisioned that the cannula comprises a fourth polymeric material, such as, for example, polycarbonate.

According to another aspect of the present disclosure, there is provided an access device, for use with surgical instruments. The access device includes a cannula made of a first material and having a distal extremity, a proximal extremity, and defines a lumen therethrough; a first collar welded to the cannula, the first collar being adapted to form a fluid tight seal around an outer perimetral surface of the cannula; and a second collar welded to the cannula at a location proximal of the first collar, the second collar being adapted to form a fluid tight seal around the outer perimetral surface of the cannula. The access device further includes a balloon made at least in part of a second material that is different from and incompatible with the first material (from which the cannula is made). The balloon is welded to the first collar and the second collar in a fluid tight manner. The one or more attachment members are made from a material that is compatible with the first and second materials, thereby facilitating securement of the balloon to the cannula.

It is envisioned that the structural balloon may include a multilayer material having a first layer of a first polymeric material, a second layer of a second polymeric material and a third layer of a third polymeric material, the second layer being interposed between the first layer and the third layer. Desirably, the multilayer material is attached to the one or more attachment members so that the first layer abuts the one or more attachment members. The cannula is made from a fourth material that is different from and incompatible with the first layer. It is envisioned that at least one of the first and third polymeric materials may be polyurethane. It is further envisioned that the second polymeric material may be a polyester. It is further envisioned that the fourth polymeric material may be a polycarbonate.

Other objects and features of the present disclosure will become apparent from consideration of the following description taken in conjunction with the accompanying drawings.

### BRIEF DESCRIPTION OF THE DRAWINGS

These and other features, aspects and advantages of the present invention will become better understood with regard to the following description, appended claims and accompanying drawings where:

FIG. 1 is a perspective view of an access device, according to an embodiment of the present disclosure;

FIG. 2 is a schematic side elevational view of an access device according to the embodiment of FIG. 2;

FIGS. 3A and 3B are schematic side elevational views of a balloon dissector assembly and the obturator portion thereof, respectively, according to the embodiment of FIGS. 1 and 2;

FIG. 4 is a schematic side elevational view illustrating the assembly of the balloon dissector assembly and access device in accordance with the embodiment of FIGS. 1-3;

FIG. 5 is a schematic side elevational view of the balloon dissector assembly and access device fully assembled, in accordance with the embodiments of FIGS. 1-4;

FIG. 6 is a cross-sectional view of a balloon attachment of the access device in accordance with the embodiment of FIGS. 1-5, taken through 6-6 of FIG. 2;

FIGS. 7A and 7B are perspective views of a collar suitable for use as an attachment member in accordance with one embodiment of the present disclosure;

FIG. 8 is a perspective view of an access device in accordance with a further embodiment of the disclosure, showing an anchor balloon deflated;

FIG. 9 is a perspective view of the access device in accordance with the embodiment of FIG. 8, showing the anchor balloon inflated;

FIGS. 10-20 are schematic illustrations showing the use of a balloon dissector and access device in accordance with a further embodiment of the disclosure;

FIGS. 21A through C show the steps in assembling the balloon assembly and the cannula in accordance with one embodiment of the present disclosure; and

FIG. 21D shows the cannula having a balloon assembly mounted thereon as part of an access device in accordance with one embodiment of the present disclosure.

### DETAILED DESCRIPTION OF PREFERRED EMBODIMENTS

Preferred embodiments of the presently disclosed surgical instrument, including an access device according to the present disclosure, will now be described in detail with reference to the drawings, in which like reference numerals designate identical or corresponding elements in each of the several views. As used herein, the term “distal”, as is conventional, will refer to that portion of the instrument, apparatus, device or component thereof which is furthest from the user while, the term “proximal”, will refer to that portion of the instrument, apparatus, device or component thereof which is closest to the user.

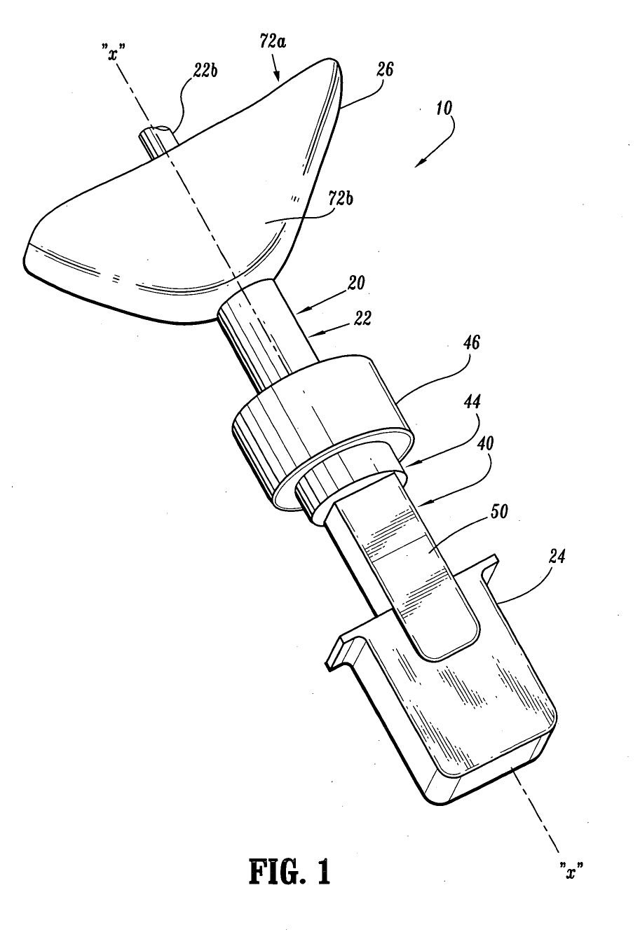

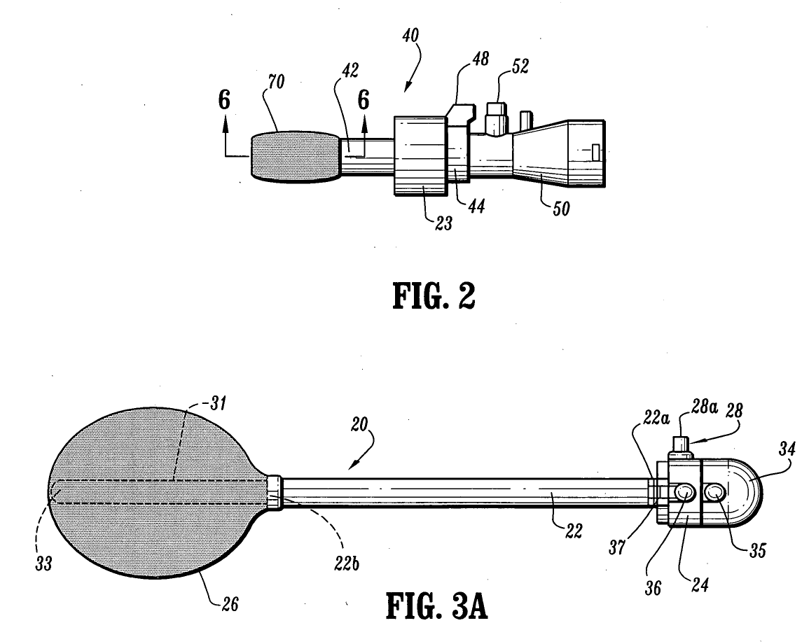

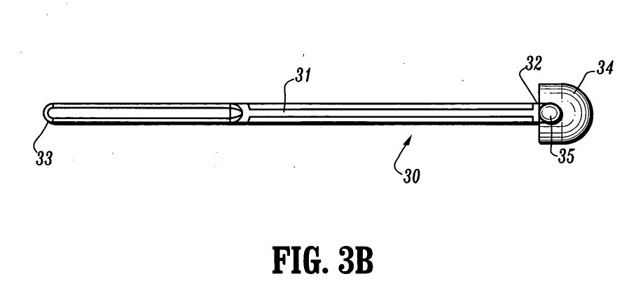

FIG. 1 shows a balloon dissector assembly 20 as used in combination with an access device 40 in accordance with the present disclosure. FIG. 2 shows the access device 40 alone and FIG. 3 shows the balloon dissector device alone. While the following disclosure relates generally to the use of access device 40 in combination with a balloon dissector assembly 20 for performing, for example, extraperitoneal hernia repair, it is envisioned and within the scope of the present disclosure that access device 40 may be used in combination with, and not limited to, balloon retractors, balloon dissectors, and the like, or any other laparoscopic surgical instrument, to perform a variety of other surgical procedures known by one having ordinary skill in the art.

Surgical dissection instruments are used for insertion into the body of a patient to create or enlarge a cavity or anatomic space. As shown in FIG. 3, balloon dissector assembly 20 includes a tubular member 22 having a bore extending therethrough, and an obturator 30 slidably mounted in the bore of the tubular member 22. The obturator 30 includes a proximal extremity 32 and a distal extremity 33 having a blunt tip. An inflatable dissection balloon 26, operatively secured to the tubular member 22, may be provided. A sleeve (not shown) may substantially enclose the collapsed balloon, or the sleeve may be omitted. The sleeve if provided, desirably has a weakened region extending longitudinally thereof, permitting the sleeve to tear or separate when the balloon is inflated in the body cavity, thereby releasing the balloon. As the balloon is inflated, the balloon creates forces, generally perpendicular to the surface of the balloon, which cause the tissue to pull apart or separate along a natural plane, providing an operating space.

The tubular member 22 has a proximal end 22a and a distal end 22b. Tubular member 22 is formed of a rigid plastic material. A housing 24 is operatively connected to the proximal end 22a of tubular member 22. The housing 24 includes a second seal member that seals the bore while the obturator 30 is disposed within the bore. Desirably, the first seal member is disposed distally from the second seal member. Reference may be made to U.S. Pat. No. 6,312,442, the entire disclosure of which is hereby incorporated herein by reference, for a more detailed discussion of the structure and use of a balloon dissector.

The dissection balloon 26 of the balloon dissector assembly 20 is operatively secured on distal end 22b of tubular member 22. The dissection balloon may have any shape and may be elastic, rigid or inelastic. In certain preferred embodiments, dissection balloon 26 advantageously may be one of two shapes (i.e., round and oval) depending on surgeon preference and patient anatomy. The dissection balloon 26 has an interior and is attached to the tubular member 22 so that the interior of the dissection balloon 26 and the bore of the tubular member 22 are in communication.

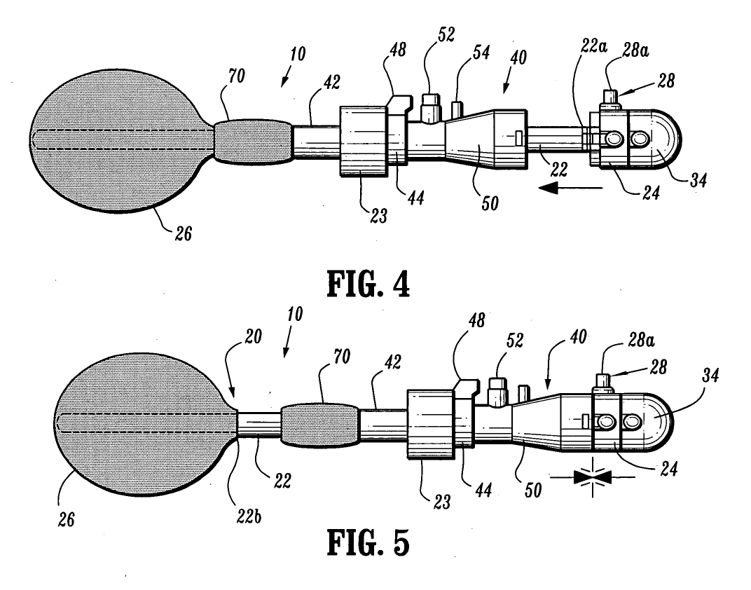

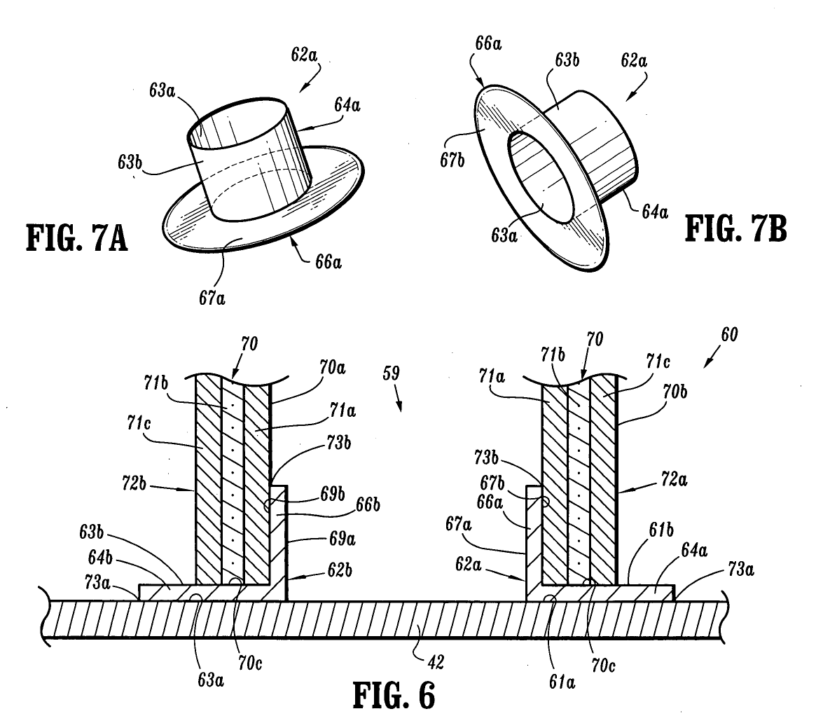

As seen in FIGS. 3-5, balloon dissector assembly 20 further includes a balloon inflation port 28, and a valve assembly 28a connected to the port 28. The valve assembly 28 couples with an inflation device 80, e.g., an inflation bulb, (see FIG. 14), for transmission of inflation fluid to dissection balloon 26. The port 28 is in communication with the bore of the tubular member 22 for utilizing inflation bulb in inflating the dissection balloon 26.

The obturator 30 comprises a shaft 31 having a proximal end 32 and a distal end 33. As best seen in FIGS. 3A, 3B and 12, a handle 34 is attached to the proximal end 32 of the shaft 31 and includes buttons 35. Buttons 35 are attached to latches (not shown) for engaging recesses (not shown) in the housing 24 so that the obturator 30 may be secured to the housing 24 to provide the balloon dissector assembly 20. Housing 24 includes buttons 36, which are also attached to latches 37 for assembly of the balloon dissector assembly 20 with the access device 40.

Turning now to FIGS. 1, 2, 4 and 5, access device 40 includes a cannula 42, a locking collar 44 operatively associated with cannula 42, and a foam collar 46 extending distally from locking collar 44. A latch assembly 48 is provided on locking collar 44 to secure the locking collar 44 to the cannula 42. Foam collar 46 is affixed to the locking collar 44 and is compressible against the abdominal wall to provide a seal. Reference may be made to International Application Serial No. PCT/US02/17359, the entire contents of which is incorporated herein by reference, for a detailed discussion of the operation and use of latch assembly 48 and foam collar 46.

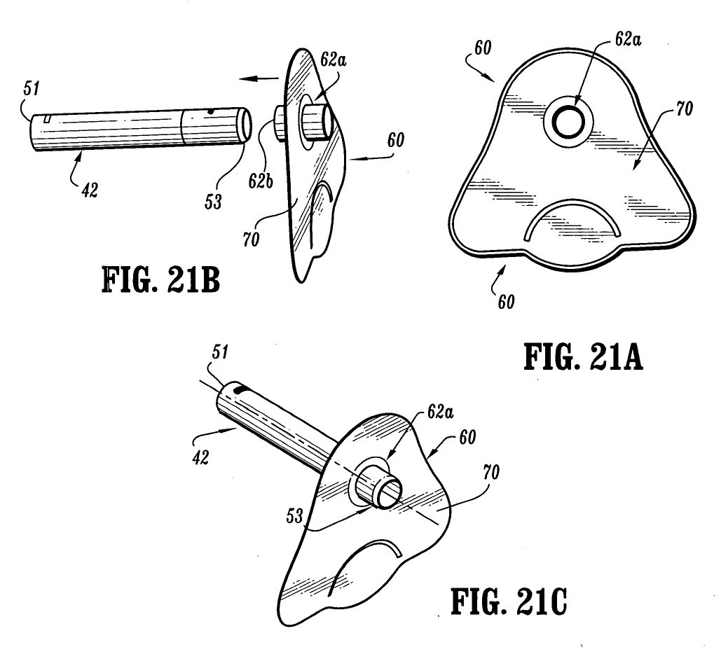

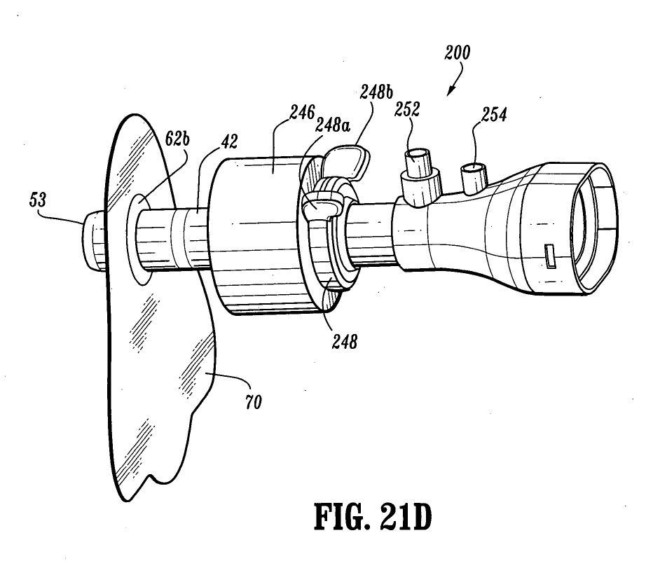

The locking collar 44 may also have a lock incorporating a torsion spring 248, in place of the latch assembly 48, as seen in FIG. 21D. The torsion spring 248 is arranged so that pressing the ends 248a, 248b of the spring together causes the spring to radially expand, allowing the user to slide the foam collar 246 along the cannula 42. When the ends of the spring are released, the position of the foam collar 246 is secured. A further device for securing the position of the access device is a skin seal having a threaded exterior. Such a device is disclosed in certain embodiments of U.S. Pat. No. 5,403,336, the disclosure of which is hereby incorporated by reference herein. In further embodiments, a rubber member is slidable along the cannula, and frictionally engages the cannula.

The cannula 42 has a proximal end 51 and a distal end 53. A housing body 50 is operatively connected to a proximal end 51 of cannula 42. Cannula 42 has a tubular wall defining a passageway communicating with an opening in the housing body 50 for receipt of operating instruments therethrough. A balloon assembly 60 is supported on or is otherwise attached to cannula 42 and is in fluid communication with an inflation port 52 provided on housing body 50. A fluid channel is defined within the wall of the cannula 42 and connects inflation port 52 with balloon assembly 60.

Cannula 42 can be made of any rigid material. Suitable material include polymeric materials. A particularly useful class of polymeric materials are polycarbonate materials.

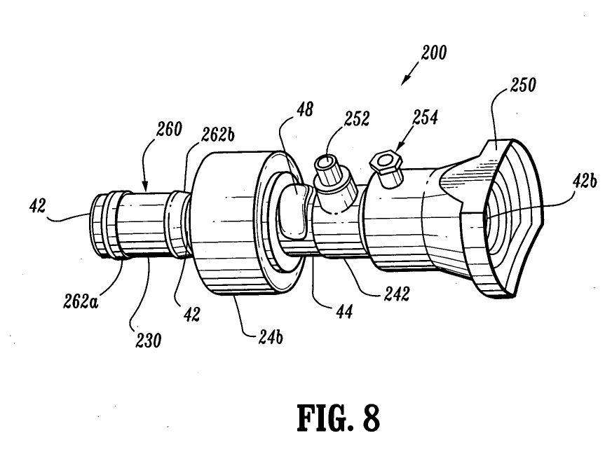

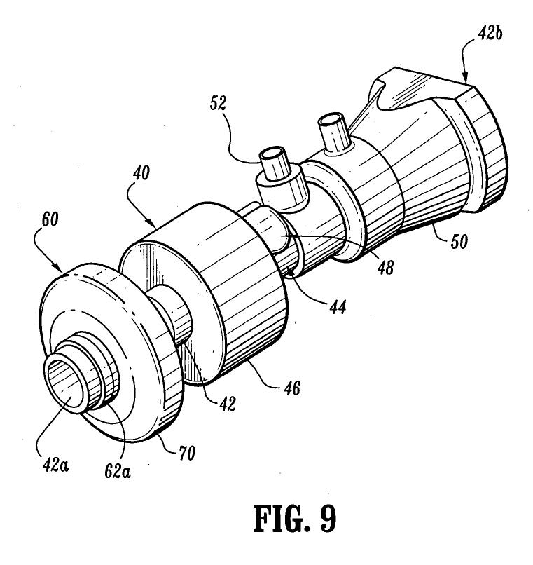

As seen in FIGS. 6 and 21A-C, balloon assembly 60 includes a pair of attachment members, namely, first or distal collar 62a and second or proximal collar 62b, each of which is attached to cannula 42. As seen in FIGS. 6, 7A and 7B, each collar 62a, 62b includes a tube portion 64a, 64b, respectively, and a flange 66a, 66b, respectively, extending orthogonally from one another. Additionally, collars 62a, 62b are positioned on cannula 42 such that respective flanges 66a, 66b of collars 62a, 62b are oriented towards one another, or are in juxtaposed relationship, and located in the interior 59 of the balloon 70. Flange 66a defines an inner surface 67a, and an outer surface 67b, and flange 66b has a inner surface 69a, and an outer surface 69b. Tube portion 64a has a cannula side 61a and a balloon side 61b, whereas tube portion 64b has a cannula side 63a and a balloon side 63b.

The collars, although shown in the figures as having a tubular shape with a generally perpendicular depending flange, may have other shapes. For example, the collars may be two separate simple cylindrical sleeves with no depending flanges. As another example, the two collars may be connected as a single sleeve with two spaced apart, depending flanges thereby forming a single attachment member. As another example, a single cylindrical sleeve with no depending flanges may be substituted for the first and second collars as a single attachment member.

The attachment members can be made using techniques within the purview of those skilled in the art. For example, the attachment members can be molded or cast from a liquid composition, such as a composition containing a polymeric material and a suitable solvent. In a particularly useful embodiment, the attachment members are formed by dipping a mandrel having an outer surface that defines the desired configuration of the attachment member into a liquid composition, such as a composition containing a polymeric material, such as CARBOTHANE®, and a suitable solvent, such as xylene. When the solvent is removed (e.g., via heating), the composition remaining on the mandrel becomes solid, can be removed and used as an attachment member. Multiple dipping and drying cycles can be performed to achieved a desired thickness for the attachment member.

Balloon assembly 60 further includes a structural balloon 70 secured to flanges 66a, 66b of collars 62a, 62b. The balloon 70 has an inner surface 70a and an outer surface 70b. In particular, structural balloon 70 is attached to collars 62a, 62b in such a manner that inner surface 70a of structural balloon 70 is secured to the outer surface 67b and 69b of respective flanges 66a, 66b of collars 62a and 62b. However, outer surface 70b may instead be attached to inner surfaces 67a and 69a of the flanges 66a, 66b. Preferably, structural balloon 70 is positioned such that an inner rim 70c of structural balloon 70 is in contact with the balloon sides 61b and 63b of tube portions 64a, 64b of collars 62a, 62b.

As seen in FIG. 6, balloon 70 preferably includes three layers, a first inner layer 71a, a second middle layer 71b, and a third outer layer 71c. In one embodiment, outer layer 71c and inner layer 71a, are fabricated from polyurethane while middle layer 71b is fabricated from polyester. It is envisioned that any number of layers may be provided. For example, structural balloon 70 may include two layers, wherein outer layer 71c is removed. Moreover, it is envisioned that layers 71a-71c may be arranged in any order. For example, it is envisioned that middle layer 71b (e.g., the polyethylene layer) is the outer layer of balloon 70. Preferably, the balloon 70 is formed from two sheets welded at the periphery and, in that case, the material of the balloon is weldable in this manner. As best seen in FIG. 1, balloon 70 further defines a distal side 72a, a proximal side 72b, and an aperture 72c (See FIG. 6) extending through distal side 72a and proximal side 72b. In certain embodiments, the distal side 72a and proximal side 72b are formed from separate sheets of material welded together at a periphery of the balloon 70. In other embodiments, the balloon 70 is formed from one or more sheets.

The material from which collars 62a, 62b are made is selected for attachment to both cannula 42 and balloon 70. For example, in one preferred embodiment, the cannula 42 comprises a polycarbonate material and the balloon 70 comprises polyurethane (and may include layers of the other materials). As those skilled in the art will appreciate, polycarbonate and polyurethane materials are difficult, if not impossible, to weld directly together. In accordance with the present disclosure, therefore, collars 62a and 62b are comprised of a material that is compatible with cannula 42 and the balloon 70 material for welding. This can be achieved, for example by forming the attachment member(s) from a polymeric material the chemical composition of which includes aspect of the two incompatible materials, either as a simple blend or chemically linked. Chemical linking can be achieved, for example, simply by forming a copolymer (e.g., a block copolymer wherein one block is composed of the polymeric material from which the balloon is made and another block is composed of the polymeric material from which the cannula is made). Those skilled in the art will envision other strategies of preparing materials that are compatible with both the material of construction of the cannula and the material of construction of the balloon. In embodiments where the balloon 70 includes a polyurethane material and the cannula 42 includes a polycarbonate material, collars 62a and 62b comprise a material that is compatible with both the polyurethane and polycarbonate materials, such as, for example, a material commercially available under the trade name CARBOTHANE® (available from Thermedics, trademark of Noveon). This material is aliphatic polycarbonate-based thermoplastic polyurethane (TPU).

Desirably, as seen in FIG. 6, a first weld 73a is provided between tube portions 64a, 64b of the respective collars 62a, 62b and cannula 42. Preferably, first weld 73a extends along the entire length of each of collars 62a, 62b. Alternatively, weld 73a is a spot or line weld formed along the proximal-most or distal-most edge of collars 62a, 62b around the entire circumference or perimeter of body portion 64 of collars 62a, 62b. Additionally, a second weld 73b is provided between balloon 70 and flanges 66a, 66b of each collar 62a, 62b. Preferably, second weld 73b extends along the entire height of annular flanges 66a, 66b. Alternatively, weld 73b is a spot or line weld formed along the radially outward-most edge of annular flange 66a, 66b around the entire circumference or perimeter of annular flanges 66a, 66b.

First weld 73a maintains the relative axial position of collars 62a, 62b with respect to cannula 42 while second weld 73b maintains the relative position of balloon 70 with respect to each collar 62a, 62b.

In a method of attaching the balloon 70 to the access device 40 a balloon assembly 60 as shown in FIG. 21A is first made. Specifically, one collar 62a is attached to the distal portion 72a of the balloon material and the other collar 62b is attached to the proximal portion 72b of the balloon material by welding the balloon material to the flange 66a, 66b for the respective collar 62a, 62b. Next, the peripheral edges of the distal portion 72a and proximal portion 72b are welded together. The balloon-collar assembly is slid onto the distal end 53 of cannula 42 and the tube portions 64a and 64b are welded to the cannula 42 as shown in FIG. 21B. It should be understood, of course that the balloon assembly 60 may be secured at the distal end 53 or may be secured at any point along cannula 42 distal of end 53, with the distal end 53 extending distally beyond the balloon 70. FIG. 21C shows the balloon assembly positioned on and secured to the cannula 42.

Turning back to FIGS. 2 and 4 and 5, housing body 50 also includes an insufflation port 54 which is in fluid communication with the interior of cannula 42 so as to provide insufflation fluid into the body of the patient. Housing body 50 has a port 52 for inflation of balloon 70. Port 52 is configured to receive an inflation nipple of inflation device “S” (see FIG. 17), so as to inflate balloon 70 of balloon assembly 60, whereas port 54 is configured for connection to a source of insufflation, as is well known in the art. The inflation device “S” may include a deflation nipple, at an opposite end of device “S” from inflation nipple.

It is envisioned and within the scope of the present disclosure that any suitable device may be connected to ports 52 and 54, and/or be integrally formed with the ports to facilitate the entry of fluid and to prevent the escape of fluid out of these ports. A check valve-type device, which may be mechanically opened, is useful for this purpose. These devices include but are not limited to a stop-cock valve (not shown) having a hose connector barb, a tubing pinch-off device or a syringe as possible connector means. Furthermore, access device 40 may include a separate desufflation button for releasing insufflation pressure through access device 40.

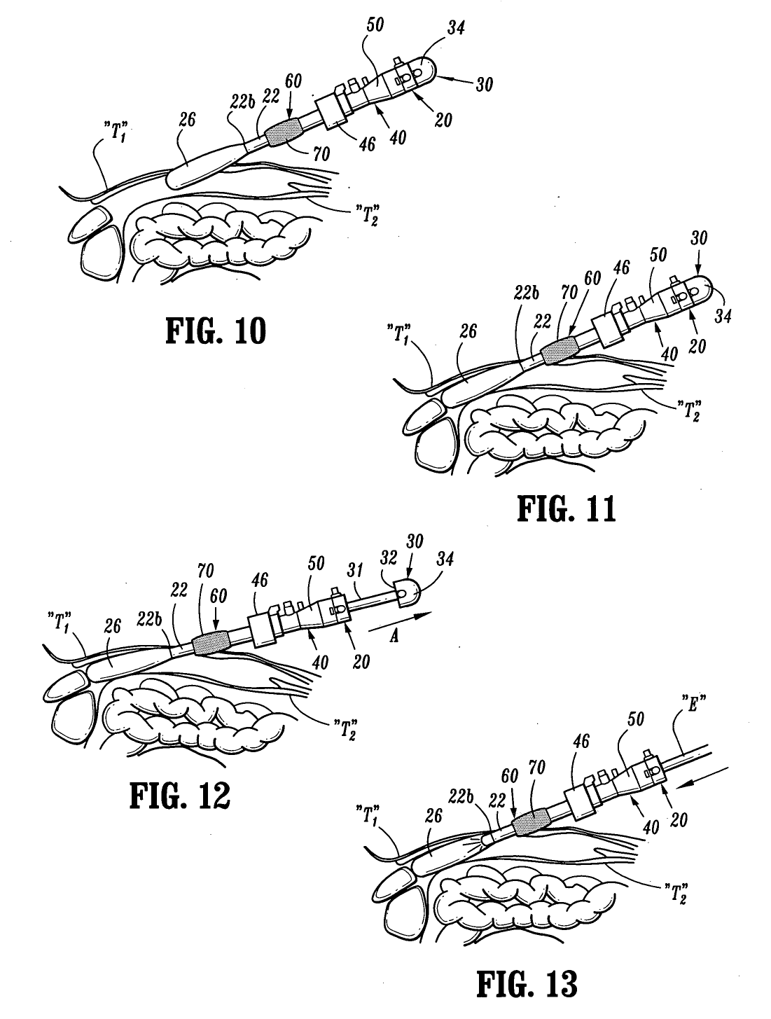

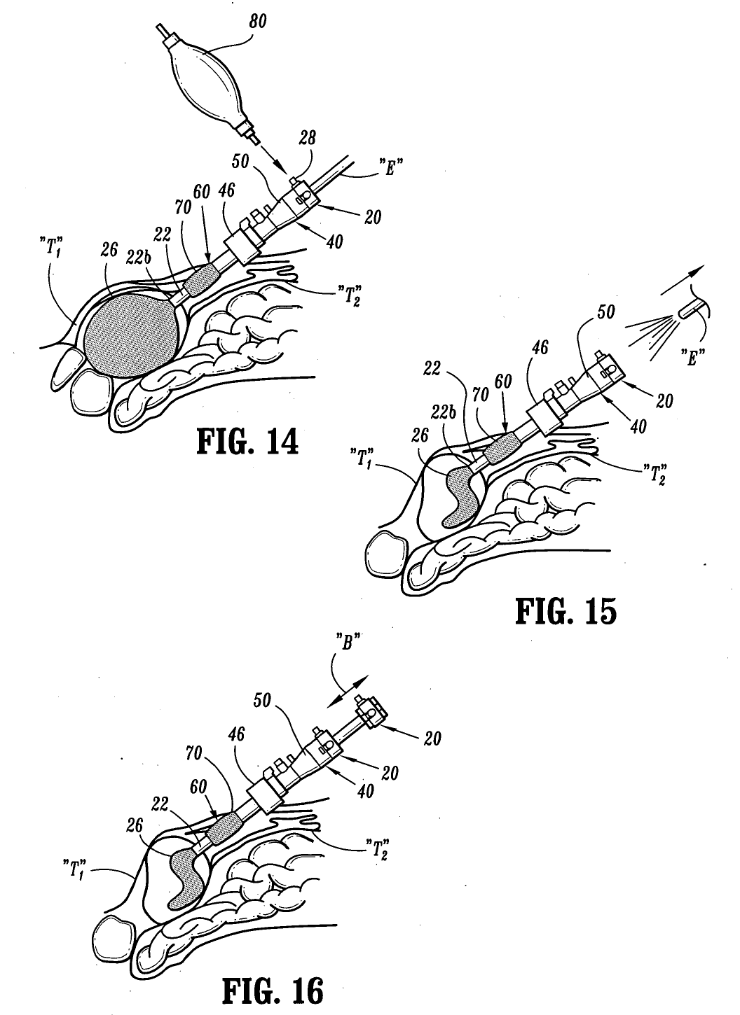

In a further embodiment of the present disclosure shown in FIGS. 8 and 9, the access device 200 comprises a generally toroidal balloon anchor 260 disposed at a distal end 242a of a cannula 242 having a housing 250. The access device 200 includes a foam collar 246 that is slidable along the cannula 242 to cooperate with the balloon anchor 260 in securing the position of the access device 200 in the patient's body. Alternatively, a threaded skin seal or rubber member may be utilized in conjunction with the balloon anchor 260, as discussed above. The housing 250, like housing 50 discussed above, has an inflation port 252 in communication with the balloon anchor 260, and an insufflation port 254 for connection to a source of insufflation gases. A passageway extends through the cannula 242, between distal end 42a and proximal end 42b, for receiving instruments being introduced into the patient's body.

The balloon anchor 260 of access device 200 may be attached to cannula 242 as discussed above in connection with FIG. 6. The balloon anchor 260 comprises a balloon 270 having the shape of a cylindrical sleeve with an aperture extending therethrough, in which the cannula 242 is to be positioned. Each of the proximal end and distal end of balloon 270 are attached to the cannula 242 through one or more collars 262, which are welded to the cannula. For example, a collar 262a for the distal end and a collar 262b for the proximal end are shown in FIG. 8. The collars 262 comprise material that is compatible with the material of the cannula 242 and the balloon 270 material for welding, whereas the materials of the cannula and the balloon 260 are not compatible, as described hereinabove.

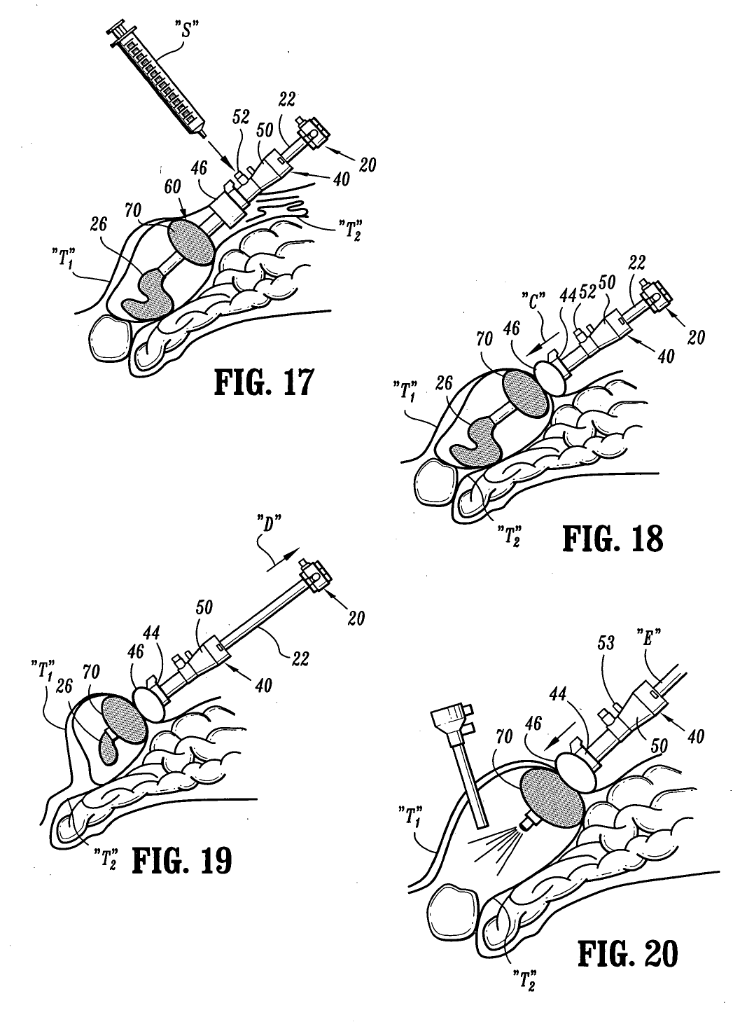

With reference to FIGS. 10-20, a method of operation and use of surgical instrument assembly 10, including balloon dissector assembly 20 and access device 40, in developing and/or maintaining an anatomic space for laparoscopic hernia repair will now be described. Initially, as seen in FIG. 10, a small incision is made in the skin of a patient, preferably in the abdominal cavity wall, in close proximity to or in the umbilicus. Preferably, with obturator 30 disposed within balloon dissector assembly 20, distal extremity 25 of cannula tube 22 is introduced into the incision, dissective between desired tissue planes T1, T2.

With distal extremity 25 of cannula tube 22 positioned in the desired location, as seen in FIG. 12, obturator 30 is withdrawn from balloon cannula assembly 20, as indicated by arrow “A”. With obturator 30 withdrawn, as seen in FIG. 13, an endoscope “E” may be inserted into dissection balloon 26 in order to visualize the inflation of dissection balloon 26. In the alternative, the obturator 30 may remain in the bore of tubular member 22 during inflation of dissection balloon 26.

As seen in FIG. 14, inflation bulb 80 is fluidly connected to valve assembly 28a of balloon dissector assembly 20. Inflation bulb 80 is then manipulated (e.g., squeezed) in order to inflate dissection balloon 26. Dissection balloon 26 is inflated until the extraperitoneal space has been sufficiently dissected.

As seen in FIG. 15, once the extraperitoneal space has been sufficiently dissected, dissection balloon 26 is deflated by removing endoscope “E” (or obturator 30) from balloon dissector assembly 20. With dissection balloon 26 deflated, as seen in FIG. 16. Latches 37 are used to detach housing 24 from the access device 40. Access device 40 is moved along tubular member 22 of balloon dissector assembly 20, as indicated by double headed arrow “B”, in order to position a distal end portion, including balloon assembly 60 of access device 40 in the incision made in the skin of the patient.

As seen in FIG. 17, an inflation instrument (e.g., a syringe “S” (as shown in FIG. 17), inflation bulb 80 (not shown) is connected to inflation port 52 of access device 40. Syringe “S” is manipulated to fully inflate structural balloon 70 of balloon assembly 60. For example, structural balloon 70 may be inflated with about 30 cc of fluid from syringe “S”. The balloon 70 is desirably formed from an inelastic material (although it may be made elastic) and shaped to maintain the operating space.

With reference to FIG. 18, foam collar 46 and locking collar 44 are slid or moved distally (e.g., in the direction of arrow “C”) along the length of cannula 42 until foam collar 46 is in contact with and is preferably compressed against the surface of the skin of the patient. Locking collar 44 is then locked in place against cannula 42. Reference may be made to International Application Serial No. PCT/US02/17359, which is hereby incorporated by reference herein, for a detailed discussion of the locking of locking collar 44.

With access device 40 locked in position against the surface of the skin of the patient (e.g., tissue “T₁”), as seen in FIG. 19, balloon dissector assembly 20 is withdrawn, in a proximal direction (as indicated by arrow “D”), from access device 40 in order to remove dissection balloon 26 from the extraperitoneal space.

As seen in FIG. 20, with balloon dissector assembly 20 removed from cannula 42 of access device 40, an insufflation fluid source (not shown) is coupled or connected to an insufflation port provided on housing body 50 of access device 40. In this manner, insufflation fluid may be delivered to the extraperitoneal space to maintain the extraperitoneal space as desired. Additionally, endoscope “E”, or other instruments, are introduced into the extraperitoneal space through cannula 42 of access device 40.

With access device 40 locked against tissue “T₁”, various surgical instruments may be introduced and withdrawn from the extraperitoneal space as needed and/or desired.

In order to remove access device 40, a deflation nozzle of the inflation bulb (not shown) may be coupled or connected to inflation port 52 and the structural balloon 70 is fully deflated. Alternatively, the access device 40 may include a deflation button on housing body 50.

Although the illustrative embodiments of the present disclosure have been described herein with reference to the accompanying drawings, it is to be understood that the disclosure is not limited to those precise embodiments, and that various other changes and modifications may be affected therein by one skilled in the art without departing from the scope or spirit of the disclosure. All such changes and modifications are intended to be included within the scope of the disclosure.

What is claimed is:

1. A surgical instrument, comprising:
a housing having an orifice;
a cannula made of a first material having a proximal end connected to the housing and a distal end, the cannula having a lumen communicating with the orifice;
a balloon made from a second material that is different from and not easily welded to the first material, the balloon having a distal side, a proximal side and an aperture extending through the distal side and the proximal side;
at least one attachment member made from a material weldable with both the first and second materials, the at least one attachment member being welded to the cannula and to the balloon.

2. A surgical device as in claim 1 wherein the at least one attachment member comprises a tubular portion.

3. A surgical device as in claim 1 wherein the at least one attachment member comprises a tubular portion and at least one depending flange.

4. A surgical device as in claim 1 wherein the at least one attachment member comprises first and second collars, each including a tubular portion and a depending flange.

5. A surgical device as in claim 1 wherein the first material is a polycarbonate.

6. A surgical device as in claim 1 wherein the second material is a polyurethane.

7. A surgical device as in claim 1 wherein the at least one attachment member is made of an aliphatic polycarbonate-based thermoplastic polyurethane.

8. A surgical device as in claim 1 wherein the first material is a polycarbonate, the second material is a polyurethane and the at least one attachment member is made of an aliphatic polycarbonate-based thermoplastic polyurethane.

9. The surgical instrument of claim 1, wherein the balloon comprises:
a multilayer material having a first layer of a first polymeric material, a second layer of a second polymeric material and a third layer of a third polymeric material, the second layer being interposed between the first layer and the third layer.

10. The surgical instrument of claim 9, wherein the multilayer material is attached to the at least one attachment member so that the first layer abuts the at least one attachment member.

11. The surgical instrument of claim 9, wherein the first polymeric material comprises polyurethane.

12. The surgical instrument of claim 9, wherein the first and third polymeric materials comprise polyurethane and the second polymeric material comprises polyester.
